(19) United States
(12) Patent Application Publication
(10) Pub. No.: US 2021/0161785 A1
(43) Pub. Date: Jun. 3, 2021

(54) Method of Preventing Both Caries and Periodontal Disease Simultaneously in One Procedure

(71) Applicant: CHX TECHNOLOGIES, INC., A CORPORATION CREATED AND EXISTING UNDER THE LAWS OF CANADA, Toronto (CA)

(72) Inventors: Julie Di Nardo, Hamilton (CA); Howard Tenenbaum, Thornhill (CA)

(73) Assignee: CHX Technologies, Inc., Toronto (CA)

(21) Appl. No.: 17/267,795

(22) PCT Filed: Aug. 13, 2019
(86) PCT No.: PCT/IB2019/001066

§ 371 Date: Feb. 10, 2021

Related U.S. Application Data
(60) Provisional application No. 62/717,586, filed on Aug. 10, 2018.

(51) Int. Cl. A61K 8/43; A61C 17/00; A61Q 11/00

(52) U.S. Cl. CPC A61K 8/43; A61Q 11/00; A61C 17/00

(57) ABSTRACT

The use of PREVORA brand topical dental solution (100 mg/ml chlorhexidine) sold by CHX Technologies, Inc., Toronto, Canada, to treat and/or prevent the advance of both dental decay (caries) and periodontal disease, simultaneously and in one procedure, that can easily be accomplished by a dental professional or a medical assistant. Chronic periodontitis and/or caries, the result of an imbalance of the microbial composition of the biofilm on the teeth, at the gum line or under the gum line, can be treated by the application of PREVORA, following standard oral cleaning and debriding of plaque at or below the gum line, to restore the balance of the biofilm to more normal, symbiotic composition. This treatment method is particularly useful in conjunction with the medical management of chronic diseases, such as type 2 diabetes, chronic obstructive pulmonary disease, cardiovascular disease, and the like, which have been associated with periodontopathogens. As an example, the topical dental solution is applied to the teeth of an adult diabetic patient every 3 months for the first year, followed by semi-annual single applications for as long as the patient remains at risk for either periodontal disease or caries.

### RELATIONSHIP TO OTHER APPLICATION

This application claims the benefit of the filing date of United States Provisional Patent Application Serial Number Ser. No. 62/717,586 filed Aug. 10, 2018, Conf. No. 2688 (Foreign Filing License Granted) in the names of the same inventors as herein. The disclosure in the identified United States Provisional Patent Application is incorporated herein by reference.

### BACKGROUND OF THE INVENTION

### Field of the Invention

This invention relates generally to a method for treating and/or preventing the advance of dental decay (caries) and periodontal disease, and more particularly, to a method for simultaneously treating and preventing both caries and periodontal disease at the same time with the same procedure conducted by a dental professional or a medical assistant.

### Description of the Prior Art

Poor oral health, as indicated by chronic periodontitis and/or caries, primarily emerges when the microbial biofilm on the teeth, at the gum line or under the gum line has a change in the balance of microorganisms. Poor oral health results from an unbalanced microbial composition wherein the mix of bacteria shifts from being symbiotic or commensal with the host to being dysbiotic. Disease-promoting bacteria then dominate the biofilm and cause poor oral health See, for example, Kilan, et al., “The Oral Microbiorne—an Update for Oral Healthcare Professionals,” Br Dent J., Vol. 221, pages 657-666 (2016).

Dysbiosis emerges most commonly with advancing age and the onset of associated chronic conditions such as Type 2 diabetes and Chronic Obstructive Pulmonary Disease (COPD); with the use of multiple medications which leads to xerostomia (dry mouth); and with conditions that cause physical limitations on maintaining good oral hygiene such as cerebrovascular disease (stroke) or Parkinson's Disease.

Oral dysbiosis involves two different types of bacteria: (1) aerobic and facultative aerobic, gram-positive bacteria above the gum line, and (2) anaerobic, gram-negative bacteria below the gum line. At the gum line, these microbial ecologies overlap.

Oral dysbiosis may occur either above or below the gum line, or in both locales at the same time. In the first circumstance, the patient experiences caries but will not necessarily have any soft tissue inflammation such as periodontal disease. In the second circumstance, the patient might have no decay but will likely have a periodontal disease. Periodontal disease is a chronic inflammation of the periodontium that manifests as bleeding upon probing by a hygienist, increasing periodontal pocket probing depth as measured by a dental professional, and/or sore gums or loose teeth as experienced by the patient.

Notably, a third circumstance exists wherein dysbiosis occurs above and below the gum line, at the same time, and in the same patient. This occurrence has been reported in at least two longitudinal studies. In one study of patients undergoing periodontal maintenance therapy, it was demonstrated that over a 4 year period, 66% of the patients experienced caries. See, Ravald, et al., “Prediction of Root Caries in Patients Treated for Advanced Periodontal Disease,”. J. Clin. Periodontal., Vol. 8, pages 400-414 (1981). This incidence is much higher than for older patients visiting the dentist but not receiving periodontal scaling. See, Hariyani, et al., “Root caries incidence and increment in the population—a Systematic Review, Meta-analysis and Meta-regression of Longitudinal Studies,” J. Dent., Vol. 77, pages 1-7 (October 2018). In a second longitudinal study over 22 years, 82% of this type of patient group (and who had advanced periodontal disease) experienced root caries. See, Reiker, et al., “A Cross-sectional Study into the Prevalence of Root Caries in Periodontal Maintenance Patients,” J. Clin. Periodontol., Vol. 26, pages 26-32 (1999).

In this context, there is both clinical merit and clinical need to manage dysbiosis occurring above and below the gum line simultaneously. Preferably, this would be accomplished with the same dental procedure to take advantage of cost-efficiencies in so doing. The dental patient need pay for only one procedure and make fewer visits to the dental practice, and the dental professional has improved productivity in terms of managing more patients and more disease per unit of time.

There is an additional healthcare or medical advantage to treating both major oral diseases simultaneously. Caries is directly linked to cardiovascular disease and to cerebrovascular disease (stroke). Root caries, and in particular, its keystone pathogen, Streptococcus mutans, are a significant modifiable risk factor for myocardial infarct. See, Nakano, et al,, “Serotype Distribution of Streptococcus Mutans a Pathogen of Dental Caries in Cardiovascular Specimens from Japanese Patients,” J. Med. Microbiol., Vol 56, Pt 4, pages 551-556 (April 2007); and Mauriello, et al., “Risk Modeling for Root Caries and Mortality in Older Adults,” IADR Abstract 896; and Lee, et al., “Tooth Loss Predicts Myocardial Infarction, Heart Failure, Stroke, and Death,” J. Dental Res., published on Jan. 22, 2019 at https://journals.sagepub.com/doi/abs/10.1177/0022034518814829. Streptococcus mutans is also a significant risk factor for cerebral micro-bleeds which are a precursor to stroke and cognitive dysfunction. See, Watanabe, et al., “Oral Cnm-positive Streptococcus Mutans Expressing Collagen Binding Activity Is a Risk Factor for Cerebral Microbleeds and Cognitive Impairment,” Sci. Rep., Vol. 6, pages 38561 (2016).

Likewise, periodontal disease and its keystone pathogen, Porphyromonas gingivalis, are closely linked to different chronic medical conditions which can overlap with cardiovascular disease and cerebrovascular disease, namely atherosclerosis (Beck, et al., “Relationship of Periodontal Disease to Carotid Artery Intima-media Wall Thickness: the Atherosclerosis Risk in Communities (ARIC) Study,” Arteriosder. Thromb. Vasc. Biol., Vol. 21, No. 11, pages 1816-1822 (Novemb 2001)); cognitive impairment and decline (Noble, et al., “Periodontitis Is Associated with Cognitive Impairment among Older Adults: Analysis of NHANES-III/” J. Neurol. Neurosurg. Psychiatry, Vol, 80, No. 11, pages 1206-1211(November 2009) and related animal studies (Illievski, et al., “Chronic Oral Application of a Periodontal Pathogen Results in Brain Inflammation, Neurodegeneration and Amyloid Beta Production in Wild Type Mice,” PLoS One, Vol. 13, No. 10, page e0204941 (Oct. 3, 2018); and Ding, et al., “Porphyromonas gingivalis, a Periodontitis Causing Bacterium, Induces Memory Impairment and Age-dependent Neuroinflammation in Mice,” Immun. Ageing., Vol. 15, page 6 (Jan. 30, 2018)); diabetes (D'Aiuto, et al., “Systemic Effects of Periodontitis Treatment in Patients with Type 2 Diabetes: a 12 Month, Single-centre, Investigator-masked, Randomised Trial,” Lancet Diabetes Endocrinol., Vol. 6, No. 12, pages 954-965 (December 2018)); and respiratory disease (Takeuchi, et al., “Periodontitis Is Associated with Chronic Obstructive Pulmonary Disease,” J. Dent. Res., Vol. 98, No. 5. Pages 534-540 (May 2019)) and Tan, et al., “Relationship Among Clinical Periodontal, Microbiologic Parameters and Lung Function in Participants with Chronic Obstructive Pulmonary Disease,” J. Periodontol., Vol, 90, No. 2, pages 134-140 (February 2019)).

Accordingly, by treating these keystone pathogens and associated pathogens in dysbiotic plaques, a physician and an associated hygienist or assistant working under the physician's direction, can perform a more comprehensive and effective chronic disease management program than otherwise would be the case were either dysbiosis for caries or dysbiosis for periodontal disease to be treated separately.

Comprehensive disease management via prevention of poor oral health (meaning both caries and periodontal disease) is vital to cost containment and value driven healthcare. See, Nasseh, et al., “The Relationship between Periodontal Interventions and Healthcare Costs and Utilization. Evidence from an Integrated Dental, Medical, and Pharmacy Commercial Claims Database;” Health Econ., Vol. 26, No. 4, pages 519-527 (Apr 2017) (Type 2 diabetes);and Jeffcoat, et al, “Impact of Periodontal Therapy on General Health: Evidence from Insurance Data for Five Systemic Conditions,” Am. J. Prey. Med., Vol. 47, No. 2, pages 166-174 (August 2014) (Type 2 diabetes; coronary artery disease; cerebral vascular disease; rheumatoid arthritis; and pregnancy). An assessment of their medical and dental claims experience by four American health insurance companies reports that medical premiums can be reduced significantly with better oral health (Avalere Health LLC, unpublished memorandum to Pacific Dental Services dated Jan. 4, 2016 titled: Evaluation of cost savings associated with periodontal disease treatment benefit).

There is, thus, a need for a method of treatment to manage dysbiosis above, below, and at the gum line, in the same dental procedure, either as part of the regular dental routine for at risk patient population or as an adjunct to conventional medical management of chronic health conditions that are associated with oral pathogens. As used herein, the term “high risk patient” refers to an individual with one or more risk factors for caries and/or periodontal disease including a recent history of these oral diseases, smoking, xerostomia, a high sugar diet, poor oral hygiene, or other chronic conditions which have bidirectional relationships with poor oral health, namely Type 2 diabetes, Chronic Obstructive Pulmonary Disease, and certain types of arthritis, such as Sjogren's Syndrome.

Dental services are normally organized and delivered in the dental practice by the hygiene team and by the dentist. Each has a different focus on and scope of practice. The hygienist essentially deals with periodontal disease and the cleaning of the teeth and removal of dental plaque both above and below the gum line. The dentist essentially deals with restoring the teeth which are decayed or broken. In this organization of the professional dental practice, treatment of both diseases by the same dental professional in the same visit does not occur.

Similarly, professional preventive dental products, and their associated procedures have been developed to treat either chronic periodontal disease or dental caries, but not both and certainly not simultaneously.

Periodontal prevention products are delivered under the gum line and are site-specific to inflamed or deepened periodontal pockets. In this regard, the instructions in the FDA drug approval packages for the following products are very clear: minocycline hydrochloride microspheres, an antibiotic marketed under the trademark Arestin by OraPharma, Inc., Bridgewater, N.J. (https://www.accessdata.fda.gov/drugsatfda_docs/nda/2001/50781_Arestin.cfm) and a biodegradable chip, which contains 2.5 mg of Chlorhexidine Gluconate sold under the trademark Periochip by Dexcel Pharma Technologies ltd, Or Akiva, Israel (https://www.accessdata.fda.gov/drugsatfda_docs/nda/98/20774.cfm).

When used in the manner specified by the FDA, neither of these two products have any documented effect on dental caries, nor are they pharmacologically or administratively likely to have any effect. Lack of anti-caries efficacy is attributed to an unsuitable, site-specific method of application (whereby localized pockets of dysbiosis are managed but newly emerging dysbiosis in other parts of the mouth, particularly those which are supra-gingival, go untreated), an inadequate strength of antimicrobial substance for whole-mouth treatment of dysbiosis, or a pharmacological inability of the antimicrobial substance to treat dysbiosis supra-gingivally.

There are also systemic antibiotics or anti-inflammatory drug products which are used to manage periodontal disease. For the former, refer to several systematic reviews: Prakazam, et al., “Antibiotics in the Management of Aggressive Periodontitis,” J. Pharm. Bioallied Sci., Vol. 4 (Suppl 2), pages S252-S255 (August 2012); Souto, et al., “Efficacy of Systemic Antibiotics in Nonsurgical Periodontal Therapy for Diabetic Subjects: a Systematic Review arid Meta-analysis,” Int. Dent. J. Vol. 68, No. 4, pages 207-220 (August 2018); Assem, et al., “Antibiotic Therapy as an Adjunct to Scaling and Root Planing in Smokers: a Systematic Review and Meta-analysis,” Braz. Oral Res., Vol. 31, page e67 (Jul. 3, 2017), For the latter, refer to the FDA approval package fora systemically delivered collagenase inhibitor consisting of a 20-mg capsule of doxycycline hyclate for oral administration that had been sold under the trademark Periostat by Collagenex, Inc., Newtown, Pa. (https://www.accessdata.fda.gov/drugsatfda_docs/nda/98/50744.cfm).

There is no evidence that these products prevent caries, nor that they are pharmacologically or administratively likely to have any effect. Systemic antibiotics and anti-inflammatory drugs are likely ineffective in managing caries for the same reasons given above, but in particular, the saliva and gingival crevicular fluid which would deliver these systemic antimicrobial substances to the tooth surface where caries occurs, can be expected to have such dilute concentrations of these substances as to be well below the bactericidal and inhibitory concentrations necessary for a treatment effect.

There is also a generic 012% chlorhexidine mouth rinse which is approved by the FDA for managing gingivitis, a precondition for, or early form of, periodontal disease. This rinse is used according to a prescription issued by the dental profession, and by the patient at home. However, there is no long-term data showing that this product is effective in moderate to severe forms of periodontal disease (see, James, et al., Chlorhexidine Mouth Rinse as an Adjunctive Treatment for Gingival Health, Cochrane Database of Systematic Review, (Mar. 31, 2017); and DaCosta, et al., Chlorhexidine Mouthwash as an Adjunct to Mechanical Therapy in Chronic Periodontitis: a Meta-analysis, JADA, Vol. 148, No. 5, pages 308-318 (2017)). Moreover, there is a controlled study showing that this product is ineffective in preventing caries in adults (see, Powell, et al., “Caries Prevention in a Community-dwelling Older Population,” Caries Res., Vol. 33, No. 5, pages 333-339 (September-October 1999)).

With regard to professional caries prevention products, such as fluoride varnish, silver diamine fluoride, high-strength fluoride dentifrice and fluoride dosage forms administered in the dental practice, none are administered in a manner that would deliver the active agent to the gum line and to the gingival crevice in sufficient concentration to have an antimicrobial effect on dysbiotic microorganisms causing periodontal disease. There is no data indicating usefulness of caries prevention products in managing periodontal disease. Moreover, none of the caries preventions products are approved for treatment and prevention of periodontal disease by any governmental authority.

Thus, there is further a need for a product and/or method of applying a product that is capable of treating dysbiosis both above and below the gum line, preferably by the same dental procedure, and that is clinically effective against both caries and periodontal disease.

### SUMMARY OF THE INVENTION

The foregoing and other objects are achieved by this invention which encompasses the use of a topical broad spectrum, high strength, sustained-release antiseptic substance to prevent caries and to treat and prevent periodontal disease simultaneously in the same patient at the same visit to a dental professional. A particularly advantageous and efficacious topical antiseptic substance foe this purpose is a dental solution of chlorhexidine (100 mg/ml chlorhexidine) sold under the trademark PREVORA by CHX Technologies, Inc., Toronto, Canada. When PREVORA is applied to the tooth surfaces, up to and including the gum line, of patients who are at high risk of either periodontal disease and/or caries, it is possible to treat and prevent inflammation and bleeding of the periodontal tissues simultaneously with the prevention of caries.

To manage dysbiosis above, below, and at the gum line in the same dental procedure, requires a special formulation of an antimicrobial substance which can “reset” the imbalance in the biofilm, and a unique method of application, namely:

A broad spectrum antimicrobial substance which is effective against both gram positive and gram negative bacteria

A concentration of this antimicrobial substance which is sufficient to penetrate and disrupt the biofilrn of microorganisms on the teeth and which has the ability by virtue of its high concentration, to stay on the surfaces of oral tissues for an extended period of time sufficient to disrupt the biofilm and delay the re-emergence of oral dysbiosis

A method to apply this formulation to all tooth surfaces and the gum line which is appropriate for its high concentration and which follows the mechanical cleaning or debridement of the existing biofilm (dental plaque) so as to minimize the biofilm's interference with the action of the compound

A method to apply this formulation which minimizes equipment, which is readily learned, which is within the scope of practice of dental hygiene or dental therapy or medical assistant (e.g., nurse or nursing assistant), and which easy to use in non-dental settings such as medical practices, pharmacies and retirement residences where high risk adults with poor oral health, without regular dental visits and with multiple chronic conditions are found

A viscosity of the formulation which enables it to penetrate the gingival crevice so as to disrupt and inhibit periodontal pathogens and which enables it to penetrate inter-proximally above the gum line

A formulation which does not impart microbial resistance or the colonization of opportunistic microbes that would cause other infections.

Chlorhexidine, which is an antiseptic and disinfectant that has bactericidal and bacteriostatic action against a wide range of gram-positive and gram-negative bacteria, is a suitable candidate for use as the pharmaceutical agent in the method of this invention. We have discovered, however, that PREVORA brand topical dental solution (100 mg/ml chlorhexidine) sold by CHX Technologies, Inc., Toronto, Canada, (herein referred to as “PREVORA”) has the all of the unique features required for the practice and implementation of this invention. Chlorhexidine is a solution of 10% (w/v) chlorhexicline, 20% (w/v) compendial-grade Sumatra benzoin, and 70% (w/v) ethanol. When applied as directed, between about 400 and 600 ml are applied to the teeth, thereby depositing about 40 to 60 mg of chlorhexidine in the oral cavity per treatment.

PREVORA involves two inter-related and sequentially applied coatings sold together as a kit: the first being an antiseptic formulation called Prevora Stage 1 and the second being an inert polymethylmethacrylate coating called Prevora Stage 2, Prevora Stage 1 serves to reduce and then inhibit the bacteria causing dysbiosis. Prevora Stage 2 serves to enhance the bonding of Prevora Stage 1 to the tooth surface and to minimize side effects such as a bitter taste or stinging of the oral mucosa associated with the application of a high strength antiseptic called Prevora Stage 1 to the gum line where keystone pathogens for both caries and periodontal disease reside. In vitro and in vivo studies show that the combination of the two coatings hereby described, deliver a bactericidal concentration to the biofilm for up to 3 days and an inhibitory concentration for several weeks.

In addition, the PREVORA formulation has a viscosity in a preferred range of about 7 to 17 cP (measured as per methods outlined in the current US Pharmacopeia). The viscosity of Prevora Stage 1 enables it to penetrate the gingival crevice so as to disrupt and inhibit periodontal pathogens and to penetrate inter-proximally above the gum line. Due to the high concentration of chlorhexicline in the product, the chlorhexidine is retained on the teeth surface for a time period sufficient to disrupt the biofilm and delay the re-emergence of dysbiosis.

Despite its efficacy, PREVORA does not impart microbial resistance or encourage the colonization of opportunistic microbes. Using Streptococcus mutans as the marker organism for microbial resistance and Candida albicans for opportunistic infections, a study of 96 adults followed over 18 months and 6 treatments with PREVORA or a placebo showed that there was no significant difference between PREVORA and the placebo in terms of microbial sensitivity between baseline and the end of study. (Source: CHX Technologies, Clinical Study #006 submitted to the European Medicines Agency, 2011, unpublished, but available upon request).

In a method of treating embodiment in accordance with the present invention, high risk adult patients, are treated for both caries and periodontal disease simultaneously by the steps of:

a) a standard professional cleaning the teeth;

b) debriding plaque at and below the gum line; and

c) applying a Prevora stage 1 topical, antiseptic solution, of chlorohexidine (100 mg/ml chlorhexidine) followed by application of Prevora stage 2 to the teeth of the patient up to and including the gum line.

As used herein, application of PREVORA means the sequential application of both Prevora Stage 1 and Prevora Stage 2 as instructed by the manufacturer.

In a treatment regimen embodiment of the present invention, a high risk adult patient is treated with PREVORA, preferably by the method steps above, with 1 to 4 applications in the first month or two following institution of the treatment. This is followed by a single semi-annual application of PREVORA for as long as the dental and/or medical professional assesses that the patient remains at risk for periodontal disease and/or caries. This risk should be assessed on at least an annual basis.

The PREVORA treatment of the present invention can be used as an adjunct to conventional medical management of chronic diseases that are associated with cariogenic and/or periodontopathic microorganisms, such as Type 2 diabetes, Chronic Obstructive Pulmonary Disease, Cardiovascular Disease, Cerebrovascular Disease (atherosclerosis), Xerostomia (dry mouth); and conditions that cause physical limitations on maintaining, good oral hygiene such as cerebrovascular disease (stroke) or Parkinson's Disease.

As a specific illustrative embodiment, a high risk adult patient having Type 2 Diabetes, can be treated with PREVORA every 3 months for the first year in conjunction with the patient's regular medical appointments for the management of diabetes, followed by a single semi-annual single application for as long as the patient remains at risk for either periodontal disease or caries.

### BRIEF DESCRIPTION OF THE DRAWING

Comprehension of the invention is facilitated by reading the following detailed description, in conjunction with the annexed drawing, in which:

FIG. 1 is a chart showing the change in probing depth measured over the course of one year for periodontal patients who were unresponsive to root planing and were treated with PREVORA brand topical dental solution (100 mg/ml chlorhexidine) in accordance with the method of the present invention.

### DETAILED DESCRIPTION

PREVORA was approved the regulatory authorities in Canada (DIN02046245), the United Kingdom (PL30669) and Ireland (PA1205), as well as by the European Medicines Agency (EMA/HJA-29/1258) as a professional product for the prevention of caries in high risk adults. These approvals are based on two controlled studies in high risk adults which showed that PREVORA significantly reduced caries over one year compared to placebo. These studies were not designed to elucidate the effect of PREVORA on periodontal disease and, in fact, none of the investigators observed or reported any effects on the periodontal tissues to the study sponsor, CHX Technologies, Inc., during or after completion of these controlled studies. See, Banting, et al, , “The Effectiveness of 10% Chlorhexidine Varnish Treatment on Dental Caries Incidence in Adults with Dry Mouth,” Gerodontology, Vol. 17, No. 2, pages 67-76 (December 2000); and Symington, et al., “Efficacy of a 10% Chlorhexidine Coating to Prevent Caries in At-risk Community-dwelling Adults,” Acta Odontol. Stand., Vol. 72, No. 7, pages 497-501 (October 2014). Therefore, the efficacy of PREVORA in the present invention was unanticipated.

From 2007 to 2018, PREVORA was only marketed in Canada, and within its approved indication which is for the prevention of root caries, an aggressive and age-associated form of caries whereby the enamel and/or cementum approximate to the cemento-enamel junction and/or the gum line are eroded via dysbiosis to expose the tooth root.

In 2007, the Province of Ontario, Canada, enabled registered dental hygienists to practice independently of the dentist. In 2013, the College of Dental Hygienists of Ontario further enabled these independent hygiene practitioners to prescribe prescription drugs if such drugs were used by the hygienist during the visit of a patient. These new laws and regulations in Ontario have restructured the conventional organization of the dental practice which was described above. The separation of professional focus on caries versus periodontal disease has become blurred in the practice of the independent hygienist. In this circumstance, the dental professional became responsible for both caries and periodontal prevention—in other words, for the whole mouth of the patient.

In 2013, one independent hygienist in Hamilton, Ontario, began to use PREVORA on patients at high risk of root caries, with the intent of using the product to prevent root caries only, as per the therapeutic indication of this drug.

This hygienist subsequently and unexpectedly observed that PREVORA markedly improved the health of inflamed and bleeding gums, an indicator of periodontal disease, when applied up to and including the gum line, as per the directions of use for caries prevention. Commonly, these patients showed marked rapid periodontal improvement within 4 weeks of the first application of PREVORA to the tooth surfaces despite the fact that PREVORA was not directly applied to periodontal tissue.

Subsequently, up to mid-2018, this same hygienist applied PREVORA to as many as 70 other adult patients with similar and consistent effect on the periodontal tissues. Most particularly, this hygienist observed a statistically significant treatment effect over one year, on the periodontal health of 8 patients who were unresponsive to the standard of hygiene care for periodontal disease—mechanical deep scaling, also referred to as scaling and root planning (“SRP”),

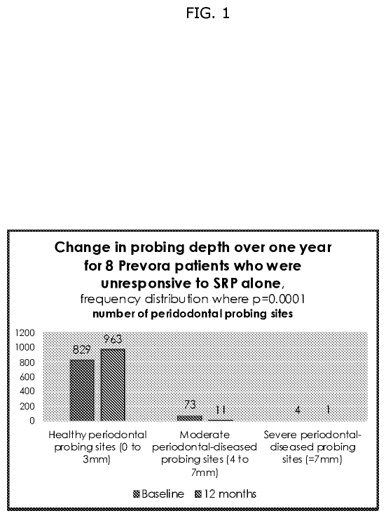

Refer to FIG. 1. Over 12 months and 5 applications administered once a week for 4 weeks, or once every 2 weeks for 8 weeks in the case of the initial four applications, followed by one single application at month 6 from the start of the treatment plan of PREVORA, the number of healthy periodontal probing sites in these unresponsive patients increased from 829 to 963 while the number of diseased sites decreased from 77 to 12.

Importantly for this discovery, only one of the 70 other patients treated in a similar fashion experienced an increase in caries during the course of applying this formulation confirming further the anticariogenic effects of PREVORA as compared to the adults experiencing both periodontal disease and root caries as reported by Ravald, et al., supra, and Reiker, et al., supra.

In view of the foregoing, it is clear that both major oral diseases have been prevented with one treatment procedure and treatment plan. As used herein, the term “simultaneously” refers to treating dysbiosis, both above, below, and at the gum line, in a manner that is capable of impacting impacts oral health, that is both periodontal disease and/or caries (particularly root caries), with one procedure that is administered to the tooth surface up to and including the gum line.

The preferred treatment plan for the simultaneous treatment and prevention of caries and periodontal disease in high risk adult patients involves up to 4 applications of PREVORA to the teeth of the patient up to and including the gum line, in the first month or two months, followed by semi-annual single applications thereafter for as long as the patient remains at risk of either disease. For adults with Type 2 diabetes who visit the physician every 3 months or so, the PREVORA treatment plan may be dovetailed with these quarterly visits in the first year. Continuation of PREVORA treatment beyond, year 1 should be accompanied by an annual risk assessment of poor oral health (defined herein as the occurrence of either caries or periodontal disease or both) involving the patient.

Since 2017, this same treatment effect on periodontal tissues has been independently and consistently observed and reported by the dental department of Mount Sinai Hospital of Toronto, Ontario with regard to adult patients with Special Needs and Complex Needs as defined as those with developmental disabilities or adults with multiple chronic diseases necessitating oral health services be conducted in a hospital setting.

The proof of caries prevention is evident from two randomized controlled clinical trials which are accepted by the following regulatory authorities in issuing drug approval of the use of PREVORA for treating dental caries: Heath Canada, the UK Medical and Healthcare Products Regulatory Agency, the Irish Health Products Regulatory Agency and the European Medicines Agency. The chart on FIG. 1 shows proof of the treatment and prevention of periodontal disease by the application of PREVORA up to and including the gum line.

No other dental product has shown the ability to manage both caries and periodontal disease simultaneously. The application of an antiseptic to the gum line so as to reduce inflammation which is below the gum line is not obvious to dental professionals who are experienced in managing such inflammation.

The use of PREVORA according to the method of this invention is advantageous to both the dental professional and the patient. Adult patients benefit from prevention of both major diseases simultaneously from the same procedure This improves the cost-effectiveness and convenience of dental services for the patient. Hygienists benefit from an expanded ability to manage both major diseases simultaneously, and are thereby able to improve their productivity and serve more patients per unit of time. This is particularly relevant to independent hygienists and dental therapists who are increasingly enabled by legislators to practice independently in Canada, the United Kingdom and the United States. Additionally, the invention minimizes the product inventory requirements and associated equipment for performing the management of caries and periodontal disease, for the dental professional. No longer is an array of products needed for the prevention of caries and, separately, for the prevention of periodontal disease.

Advantageously, the application of Prevora Stage 1 and Stage 2 requires only a small compressor and connected hand syringe to clear the teeth and gingival crevice, and to dry the teeth prior to the application of PREVORA. PREVORA is applied with a mini-brush common to dental hygiene practices, with separate brushes being used for the application of Prevora Stage 1 and Prevora Stage 2. This equipment set up is suitable for venues such as the medical examination room, the wellness center in retirement residences and the consultation rooms in pharmacies

Although the invention has been described in terms of specific embodiments and applications, persons skilled in the art may, in light of this teaching, generate additional embodiments without exceeding the scope or departing from the spirit of the invention described and claimed herein. Accordingly, it is to be understood that the drawing and description in this disclosure are proffered to facilitate comprehension of the invention, and should not be construed to limit the scope thereof.

1. The use of a topical antiseptic dental solution of chlorhexidine (100 mg/ml chlorhexidine) sold under the trademark PREVORA by CHX Technologies, Inc., Toronto, Canada, applied to the tooth surfaces of patients at high risk of either periodontal disease and/or caries up to and including the gum line to treat and prevent inflammation and bleeding of the periodontal tissues simultaneously with the prevention of caries.

2. A method of treating high risk adult patients for both caries and periodontal disease simultaneously comprising the steps of:
a) cleaning the teeth;
b) debriding plaque at and below the gum line; and
c) applying a topical antiseptic solution of chlorhexidine (100 mg/ml chlorhexidine) sold under the trademark PREVORA by CHX Technologies, Inc., Toronto, Canada, to the tooth surfaces of the patient up to and including the gum line.

3. The use of a topical antiseptic solution according to claim 2 wherein the PREVORA brand topical dental solution is applied to said high risk adult patients in a treatment regimen that involves from 1 to 4 applications of PREVORA brand topical dental solution in the first month or two of instituting treatment followed by a single semi-annual application of PREVORA brand topical dental solution for as long as the patient remains at risk for either periodontal disease or caries.

4. The use of a topical antiseptic solution according to claim 3 comprising the step of assessing the risk for either periodontal disease or caries on an annual basis.

5. The method of claim 3 wherein the high risk adult patient has Type 2 Diabetes and the PREVORA brand topical dental solution is applied every 3 months for the first year in conjunction with medical appointments for the management of diabetes, followed by a single semi-annual single application for as long as the patient remains at risk for either periodontal disease or caries.

6. The use of a topical antiseptic solution applied to the tooth surfaces up to and including the gum line to treat and prevent inflammation and bleeding of the periodontal tissues simultaneously with the prevention of caries in the management of chronic disease that are associated with cariogenic and/or periodontopathic microorganisms.

7. The use of a topical antiseptic solution in accordance with claim 6 wherein the chronic disease is selected from the group consisting of Type 2 diabetes, Chronic Obstructive Pulmonary Disease, Cardiovascular Disease, Cerebrovascular Disease (atherosclerosis), Xerostomia (dry mouth); and conditions that cause physical limitations on maintaining good oral hygiene such as cerebrovascular disease (stroke) or Parkinson's Disease.

8. The use of a topical antiseptic solution according to claim 6 wherein the antiseptic solution is PREVORA brand topical dental solution (100 mg/ml chlorhexidine) sold by CHX Technologies, Inc., Toronto, Canada.

9. The use of a topical antiseptic solution according to claim 7 wherein the antiseptic solution is PREVORA brand topical dental solution (100 mg/ml chlorhexidine) sold by CHX Technologies, Inc., Toronto, Canada.
